(19) United States
(12) Patent Application Publication
(10) Pub. No.: US 2022/0279933 A1
(43) Pub. Date: Sep. 8, 2022

(54) Cooling Pad Device

(71) Applicant: Bobbi Sommerman, Joppa, MD (US)

(72) Inventor: Bobbi Sommerman, Joppa, MD (US)

(21) Appl. No.: 17/674,008

(22) Filed: Feb. 17, 2022

Related U.S. Application Data
(60) Provisional application No. 63/158,059, filed on Mar. 8, 2021.

(51) Int. Cl. A47C 21/04

(52) U.S. Cl. CPC A47C 21/046

(57) ABSTRACT

The present invention relates to a cooling pad device that is comprised of a cooling filling, an internal pad, and a cover. The body of the device further preferably resembles the size and dimensions of any mattress size or shape for any age or demographic and is comprised of a flexible or semi-flexible material. The device can be attached to a fitted bed sheet by at least one fastener, and upon weight activation, emit a cold temperature from internal gel packets to reduce fever. In differing embodiments, the cover of the device may have a plurality of indicia such as but not limited to: patterns, logos, emblems, images, symbols, designs, letter, number, words, characters, animals, advertisements, brands, etc.

### CROSS-REFERENCE TO RELATED APPLICATION

The present application claims priority to, and the benefit of, U.S. Provisional Application No. 63/158,059 which was filed on Mar. 8, 2021, and is incorporated herein by reference in its entirety.

### FIELD OF THE INVENTION

The present invention relates generally to the field of sleeping pads. More specifically, the present invention relates to a cooling pad device that is comprised of a cooling filling, an internal pad and a cover. The body of the cooling pad device preferably resembles the size and dimensions of any mattress size or shape used by any age or demographic, and is comprised of a flexible or semi-flexible material. The cooling pad device can be attached to a fitted bed sheet by at least one fastener and, upon weight activation, emits a cold temperature or coolness from internal gel packets to reduce a user's fever. In differing embodiments, the cover of the cooling pad device may have a plurality of indicia thereon such as, but not limited to, patterns, logos, emblems, images, symbols, designs, letter, number, words, characters, animals, advertisements, brands, etc. Accordingly, the present disclosure makes specific reference thereto. Nonetheless, it is to be appreciated that aspects of the present invention are also equally applicable to other like applications, devices, and methods of manufacture.

### BACKGROUND

High fevers requiring the attention of a caregiver can often occur in infants, toddlers, and the elderly—individuals unable to adequately care for themselves when ill. Caregivers may be responsible for not only attending to the needs of said individuals by administering medications, feeding and checking in, but also cooling down the temperature of the fever itself should the need arise. Children under the age of two years old are particularly subject to harmful neurological or immunological damage should their fever reach 104 degrees Fahrenheit of higher. As a result, continual monitoring and management of body temperature is critical for a caregiver to nurse his or her child, loved one or patient responsibly and safely. Unfortunately, it is common for fevers to spike during the nighttime, when caregivers are likely sleeping and therefore inattentive. This means a caregiver must wake himself or herself up continually throughout the night to check on his or her child, loved one or patient, or the child's, loved one's or patient's health may be at risk from a spike in fever. Waking up throughout the night may be exhausting, and lead to a decrease in performance in overall caregiving, thereby potentially risking further harm to the child, loved one or patient.

Therefore, there exists a long-felt need in the art for an improved cooling pad device. There also exists a long-felt need in the art for a cooling pad device that may supply cooling temperatures to ill children or elderly people that are not able to care for themselves. Further, there exists a long-felt need in the art for a cooling pad device that maintains a cool temperature throughout the night, such that a caregiver need not replace a cold cloth, towel, fabric, or icepack continuously throughout the night. In addition, there exists a long-felt need in the art for a cooling pad device that is comfortable to lay on and can be secured beneath the fitted sheet of a mattress for an ill person to sleep without disturbance.

The subject matter disclosed and claimed herein, in one embodiment thereof, comprises a cooling pad device. The device is primarily comprised of an internal pad and a cover. The body of the device further preferably resembles the dimensions of any size mattress, including but not limited to; a bassinet, crib, play pen, twin bed, double bed, queen bed, and king bed, and is rectangular in shape with moderate thickness such that it does not disturb the sleep or comfort of the user. The body of the device is comprised of flexible or semi-flexible material such that it does not get bunched under the user during sleep, and may be inserted beneath the fitted sheet that typically extends over the edges of a mattress. The interior of the device is comprised of a cooling gel that is activated upon detection of a user's weight and is contained within waterproof encasing to prevent leaks. In this manner, the cooling pad device can replace the need for a caregiver to constantly monitor the need for continued application of cold towels, compresses, etc.

In this manner, the cooling pad device of the present invention accomplishes all the forgoing objectives and provides an improved means to care for a sick person with a fever. Further, the device is situated comfortably beneath the fitted sheet of a mattress as to not allow it to bunch beneath the user or cause discomfort during use. In addition, the device can be activated in its cooling properties by the weight of the person, allowing for the proportional amount of coolness to be applied that corresponds to the size of the individual. The device is also contained within waterproof encasing, such that it may prevent leaks.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a cooling pad device. The device is primarily comprised of a weight sensitive cooling filling, an internal pad, and a cover such that a caregiver need not replace cold packs and compresses throughout the night to a fevered person. In differing embodiments, the device may have the appearance of a plurality of shapes and sizes but is preferably manufactured in such a way that may meet the dimensions of any mattress appropriate for any age group or demographic. Additionally, the device may be manufactured with a variety of cooling level abilities which may subsequently alter the thickness of the device. The device may be manufactured from a variety of materials making it comfortable for use underneath a fitted mattress sheet and may be fastened to the sheet as to prevent bunching and wrinkling during the course of the night. These fasteners may be simple in function so that a user may be able to remove the fasteners themselves throughout the night should their fever break, and the material of the internal pad be lightweight so that it may be easy to lift and remove from a bed. Additionally, the body is preferably comprised of a flexible or semi-flexible soft material. Further, the outer surface of the cover may be comprised of a plurality of indicia such as patterns, logos, emblems, images, symbols, designs, letters, words, characters, animals, advertisements, brands, etc. that may or may not be fever or coolness related.

In one embodiment, the internal pad has a fixedly or removably attached cooling pack system wherein at least one weight-activated gel pack may be removed from the interior of the internal pad. The gel pack is contained within an internal pad that is manufactured from a comfortable material that is able to circulate coolness from the gel packs. The pack is preferably contained within the internal pad such that they may not freely move around but is fixed in place as to ensure there is not an overconcentration of cold packs in one area and an absence of packs in a differing area.

Accordingly, the cooling pad device of the present invention is particularly advantageous as it awards a caregiver the opportunity to sleep through the night with the knowledge that their child, loved one or patient with a fever is receiving continual cold temperatures from a cooling pack to keep their fever down. Further, the device can be universally laid upon any mattress in such a manner that does not require unique tailoring or maintenance to one specific bed shape. Additionally, the device is easily fastened and unfastened, and gel packs may be removed so that the exterior cover and or internal pads may be washed and reused. Therefore, the device eliminates the need for a caregiver to constantly check up on their child, loved one or patient throughout the night to change cold compresses and allows for the delivery of cold compresses to be continual, automatic, weight sensitive, and thus proportional in intensity to the size of the user. In this manner, the cooling pad device overcomes the limitations of existing fever reducing tools known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a cutaway view of one potential embodiment of a cooling pad device of the present invention in accordance with the disclosed architecture;

FIG. 2 illustrates a cross-sectional view of one potential embodiment of a cooling pad device of the present invention in accordance with the disclosed architecture; and

FIG. 3 illustrates a cross-sectional view of one potential embodiment of a cooling pad device of the present invention while being used in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved fever reducing, cooling pad device. There also exists a long-felt need in the art for a fever-reducing cooling pad device that is positioned comfortably under the fitted sheet of a mattress as to not induce uncomfortable bunching during use. Further, there exists a long-felt need in the art for a cooling pad device that is comprised of a cooling gel such that its cooling properties are activated upon the weight of the user, allowing a level of coolness to be applied that is directly proportional to the size of the user. In addition, there also exists a long-felt need in the art for a cooling pad device that is comprised of waterproof encasing as to prevent leaks. Also, there exists a long-felt need in the art for a cooling pad device that eliminates the need for a caregiver to provide constant attention to their ill child, loved one or patient, so that they may receive rest, and their ill child, loved one or patient may not suffer the consequences of accidental mistakes from their caregiver as a result of fatigue from providing constant care.

The present invention, in one exemplary embodiment, is comprised of a cooling pad device that allows a caregiver to sleep during the night, and an ill person requiring the exchange of cooling pads or compresses to receive continued cooling application throughout the night. The device is primarily comprised of a gel filling, an internal pad, and a cover. In differing embodiments, the device may also have the appearance of a plurality of different shapes of bed, including but not limited to: a bassinet, play pen, crib, twin bed, double bed, queen bed, and/or king bed. The device may also be made of a plurality of flexible or semi flexible materials that may be commonly used for gel insulation. In addition, the surface of the outer surface of the cover may be comprised of a plurality of indicia such as patterns, logos, emblems, images, symbols, designs, letters, words, characters, animals, advertisements, brands, etc. that may or may not be sleeping or fever related.

Further, the device has an internal pad which is comprised within the top, bottom, and side surfaces. In one embodiment the pad and/or outer surface may have at least one fastener that allows the device to attach to a fitted sheet and/or mattress to prevent wrinkling and bunching beneath the user during sleep. The internal pad has a gel filling that emits a cool temperature upon the weighted pressure of the individual laying down on it. Furthermore, the pad is preferably made from a waterproof material to prevent leaks of the gel filling.

Accordingly, the cooling pad device of the present invention is particularly advantageous as it allows a caregiver to sleep through the night while their child, loved one or patient may receive continual cooling temperatures, aiding in the reduction of fever temperatures. Further, the device can be universally positioned beneath any fitted bed sheet of any size, shape, or manner, and remain fastened throughout the night to prevent wrinkling and bunching under the user. Additionally, the device is comprised of a weight sensitive cooling gel, to emit a cooling temperature in an intensity that is proportional to the weight of the user.

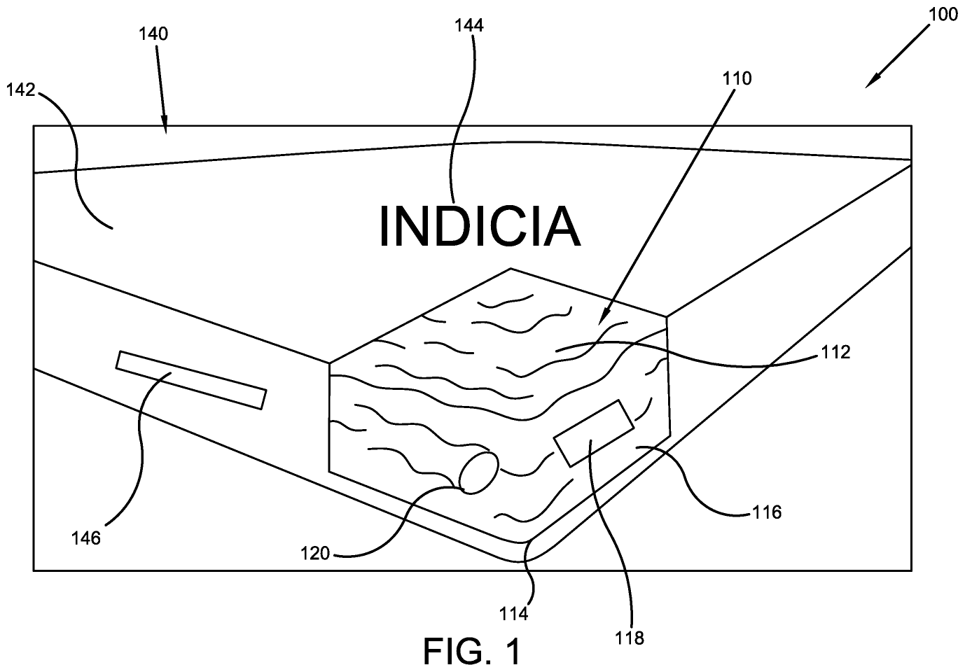

Referring initially to the drawings, FIG. 1 illustrates a cutaway view of one potential embodiment of a cooling pad device 100 of the present invention in accordance with the disclosed architecture. The device 100 is primarily comprised of an internal pad 110 and a cover 140. In differing embodiments, the device 100 may be any shape or size appropriate to fit a variety of mattresses 14, including but not limited to: a bassinet, play pen, a crib, a twin bed, a double bed, a queen bed, a king bed., etc. The device 100 may be placed and secured underneath a fitted sheet 12 via at least one fastener 146. The fastener 146 may be any fastener known in the art such as, but not limited to: adhesive, magnetic, hook and loop, snap-button, ties, etc. that allows the cover 140 to be secured to fitted sheet 12. The fastener 146 may also allow the cover 140 to be removed from around the pad 110, wherein the fastener 146 is in the form of a zipper, snap-button, tie, hook and loop, etc. Further the top surface 112, bottom surface 114, and side surfaces 116 of the pad 110 may be made from a waterproof material to prevent leaks of the interior filling 132 and condensation build up, and may be manufactured from a plurality of materials, including but not limited to: polyurethane laminate, rubber, vinyl, polyesters, wool, etc. The top surface 112, bottom surface 114, and side surfaces 116 may additionally be secured to the cover 140 of the device 100 via at least one fastener 118. The fastener 146 may be any fastener known in the art such as, but not limited to: adhesive, magnetic, hook and loop, snap-button, ties, etc.

The cover 140 of the device 100 may be fixedly or removably attached around the pad 110 and may be manufactured with a variety of materials in differing embodiments, including but not limited to canvas, silk, cotton, polyester, wool, or other plastic and fabric material of the like. The cover 140 of the device 100 may feature any number of indicia 144 in the form of patterns, logos, emblems, images, symbols, designs, letters, words, characters, animals, advertisements, brands, etc. that may or may not be sleep or fever related. The internal pad 110 of the device 100 in differing embodiments may also be manufactured from a material such as memory foam or yarn, which may serve to increase the comfort of the user 10 while they lay upon the device 100.

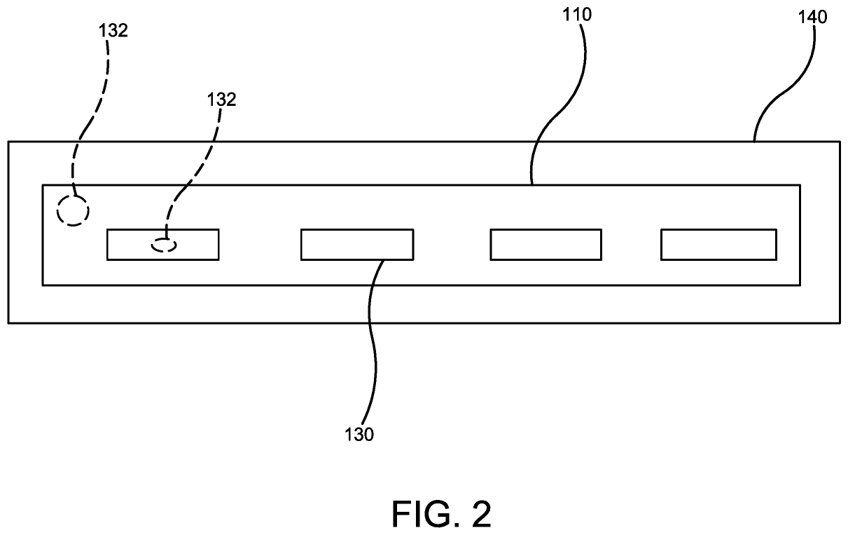

FIG. 2 illustrates a front perspective view of one potential embodiment of the cooling pad device 100 of the present invention. The device 100 contains within it the filling 132, wherein the filling 132 is preferably a weight sensitive cooling gel. The filling 132 activates cold temperatures unto the user 10 when said user 10 lays upon the top surface 112 of the device 100. The weight sensitive, cooling gel filling 132 of the device 100 may be manufactured from plurality of materials such as, but not limited to: hydroxyethyl cellulose, sodium polyacrylate, vinyl-coated silica gel, etc.

In one embodiment, the filling 132 may be freely dispersed throughout the interior of the internal pad 110. In a differing embodiment, the filling 132 is wrapped in at least one pack 130, and then embedded within the internal pad 110. The pack or packs 130 may be fixedly or removably attached within the pad 110, such that a user 10 may be able to wash the internal pad 110, surfaces 112, 114 and 116, as well as the cover 140 by first removing the packs 130. The internal portion of the pad 110 may be accessed by at least one fastener 118 such as, but not limited to: zipper, snap-button, hook and loop, ties, etc.

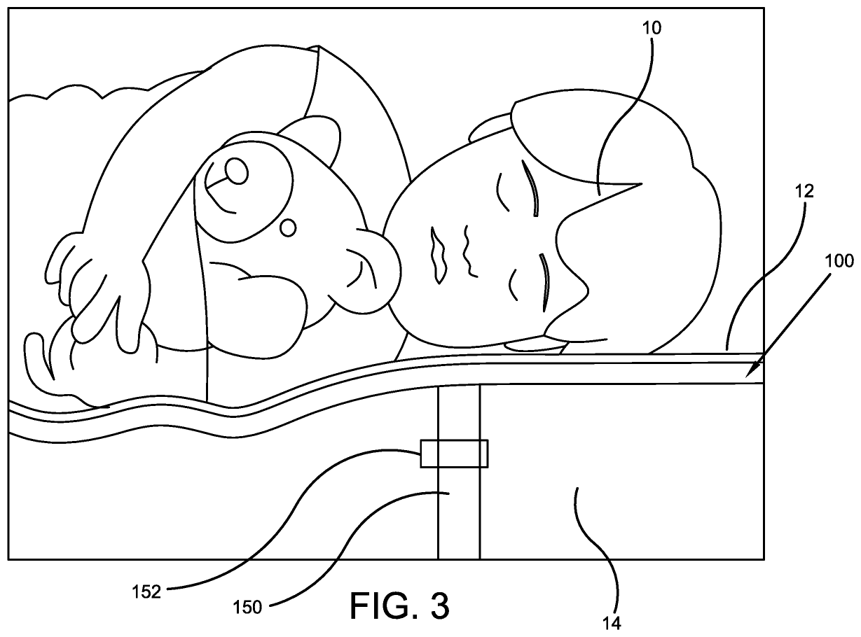

FIG. 3 illustrates a side perspective view of one potential embodiment of the cooling pad device 100 of the present invention as a user 10 lays upon it in accordance with the disclosed architecture. The user 10 is shown to be laying first upon a fitted sheet 12 which is stretched over the device 100 and fitted over the corners of a mattress 14. As shown, the device 100 is manufactured from a flexible or semi flexible material as to provide some indentation or conformity to the user's 10 body while they sleep comfortably. Additionally, the device 100 may have a plurality of thickness levels which may correspond to the amount of cooling gel filling 132 within the device 100, offering differing grades of cooling and cushioning potential depending on the needs of the user 10. The device 100 may also be comprised of at least one strap 150. The strap 150 can be placed around a mattress 14 in order to secure the device 100 to the mattress 14 and prevent shifting of the device 100 during use. The strap 150 is preferably manufactured from a fabric and/or elastic material. The strap 150 may also have at least one fastener 152 that allows a user to tighten or loosen the strap 150 around a mattress 14. Said fastener 152 may include, but is not limited to: snap-button, magnet, hook and loop, ties, adjustable buckle, sliding fastener, etc.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “cooling pad device” and “device” are interchangeable and refer to the cooling pad device 100 of the present invention.

Notwithstanding the forgoing, the cooling pad device 100 of the present invention and its various components can be of any suitable size and configuration as is known in the art without affecting the overall concept of the invention, provided that it accomplishes the above-stated objectives. One of ordinary skill in the art will appreciate that the size, configuration and material of the cooling pad device 100 as shown in the FIGS. are for illustrative purposes only, and that many other sizes and shapes of the cooling pad device 100 are well within the scope of the present disclosure. Although the dimensions of the cooling pad device 100 are important design parameters for user convenience, the cooling pad device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cooling pad device comprising:
an internal pad;
a filling; and
a cover.

2. The cooling pad device of claim 1, wherein the filling is a cooling gel.

3. The cooling pad device of claim 2, wherein the filling is positioned within the internal pad.

4. The cooling pad device of claim 1, wherein the cover encloses the internal pad.

5. The cooling pad device of claim 1, wherein the internal pad is waterproof.

6. A cooling pad device comprising:
an internal pad comprised of a first fastener;
a gel filling positioned within the internal pad; and
a cover that surrounds the internal pad and that is further comprised of a second fastener.

7. The cooling pad device of claim 6, wherein the first fastener allows the internal pad to be secured to the cover.

8. The cooling pad device of claim 6, wherein the second fastener allows the cover to be secured around the internal pad.

9. The cooling pad device of claim 8, wherein the cover is removable from the internal pad.

10. The cooling pad device of claim 6, wherein the gel filling is dispersed throughout an interior of the internal pad.

11. A cooling pad device comprising:
an internal pad;
a first fastener;
a pack;
a gel filling contained within the pack;
a cover that is sized and configured to encase the internal pad; and
a second fastener.

12. The cooling pad device of claim 11, wherein the pack is located within the internal pad.

13. The cooling pad device of claim 12, wherein the pack is removable from the internal pad.

14. The cooling pad device of claim 11, wherein the gel filling is weight-activated.

15. The cooling pad device of claim 14, wherein the gel filling emits a cold temperature.

16. The cooling pad device of claim 15, wherein the gel filling is manufactured from a group consisting of a hydroxyethyl cellulose, a sodium polyacrylate and a vinyl-coated silica gel.

17. The cooling pad device of claim 11, wherein the internal pad is manufactured from a waterproof material.

18. The cooling pad device of claim 11, wherein the cover comprises an outer surface having an indicia thereon, and further wherein said indicia is at least one of a pattern, a logo, an emblem, an image, a symbol, a design, a letter, a word, a character, an animal, an advertisement and a brand.

19. The cooling pad device of claim 11, wherein the internal pad is manufactured from a memory foam material.

20. The cooling pad device of claim 11, wherein the cover is removable from the internal pad.
